(19) United States
(12) Patent Application Publication
(10) Pub. No.: US 2004/0110781 A1
(43) Pub. Date: Jun. 10, 2004

(54) Pharmaceutical compositions containing indistinguishable drug components

(76) Inventors: Troy M. Harmon, Lansdale, PA (US); Gopi M. Venkatesh, Vandalia, OH (US)

Correspondence Address: THOMPSON HINE L.L.P., 2000 COURTHOUSE PLAZA , N.E., 10 WEST SECOND STREET, DAYTON, OH, 45402, US

(21) Appl. No.: 10/310,357

(22) Filed: Dec. 5, 2002

(51) Int. Cl. A61K 31/485; A61K 9/26; A61K 9/14

(52) U.S. Cl. 514/282; 424/469

(57) ABSTRACT

An oral pharmaceutical multi-particulate dosage form comprising at least two populations of particles, a first population of opioid agonist particles and a second population of opioid antagonist particles and a method for producing the dosage form are described. The opioid agonist particles provide an analgesically effective amount of opioid agonist and the opioid antagonist particles provide an amount of opioid antagonist effective to attenuate side effects associated with chronic dosing of the opioid agonist. The first population of opioid agonist particles and the second population of opioid antagonist particles are visually indistinguishable, thereby reducing the potential for drug abuse of the opioid agonist by separation of the two particle populations. In accordance with particular aspects of the invention, the opioid agonist particles and opioid antagonist particles are each provided with sustained release membrane coatings capable of releasing the opioid agonist and opioid antagonist at proportionate rates over a dosing interval. The dosing interval may be at least about 8 hours and preferably from about 12-24 hours. The dosage form in accordance with particular embodiments of the present invention may further comprise a non-opioid analgesic, preferably in the form of immediate release or sustained release particles.

### BACKGROUND

Morphine and other opioid antagonists have generally been considered to produce primarily inhibitory effects on nerve cells. These opioid inhibitory effects selectively block transmission of pain signals into the CNS and underline the clinical use of morphine and related opioids as analgesics. However, the chronic use of these opioids leads to tolerance to and dependency on these opioids, and the subsequent removal of the drug often precipitates aversive withdrawal syndromes. Opioid tolerance and dependency play important roles in drug addiction and since their mechanisms of action have not been clearly understood, these problems continue to seriously impede clinical treatment of chronic pain patients suffering from progressive cancer, pulmonary diseases, degenerative joint disease and chronic abdominal pain, with morphine or other opioid analgesics. Psychological dependence (i.e., addiction) on opioids is characterized by drug-seeking behavior directed towards achieving euphoria, and escape from, e.g., psychosocio-economic pressures. An addict will continue to administer opioids for non-medical purposes and in the face of self-harm, particularly self administering parenterally, the drug being more potent than when dosed orally.

There have been attempts to control the abuse potential associated with opioid analgesics and also, to provide safe, effective and practical means for alleviating the serious complication of intestinal hypomotility in chronic pain and cough patents undergoing opioid therapy. U.S. Pat. No. 3,493,657 to Lewenstein discloses that naloxone is useful for parenteral administration in conjunction with opioid agonists for ablating the respiratory depression caused by opioid agonists. U.S. Pat. No. 4,769,372 to Kreek discloses the use of specific opioid antagonists, such as naloxone, to counteract the intestinal hypomotility provoked by long term administration of opioid agonists while not blocking or interfering with any systemic agonist activity. Kreek also provides detailed dosage ranges and ratios for opioid analgesics/antitussives and suitable antagonists, which must be orally co-administered in order to be therapeutically effective.

In the management of pain in patients suffering from chronic diseases, considerable inter-subject variability in the response to a given dose of a given opioid agonist has been observed, and therefore, considerable variability among patients in the analgesic dosage required to control pain (about eight-fold range in the daily dose to control pain in approximately 90% of patients) without unacceptable side effects has been observed. U.S. Pat. Nos. 4,861,598, 4,990,341, 5,266,331, and 5,549,912 (all to Oshlack et al. with detailed dosage ranges and ratios for opioid analgesics to suitable antagonists) disclose controlled release opioid agonist formulations, which have substantially less inter-subject variation with regard to the dosage of opioid analgesic required to control chronic pain without unacceptable side effects (approximately four-fold range for a 12-hourly dose). Both 12-hourly and once-daily products containing morphine are available in the market. For example, MS Contin®, Kapanol® and Oramorph® (Roxanol®), all exhibit flattened serum profiles and the once-daily capsule disclosed in U.S. Pat. No. 5,478,577 provides a relatively large peak to trough concentration profile.

In the case of the combinations of opioid agonists and antagonists, controlled release formulations are proposed in order to reduce the frequency of medication thereby improving patient compliance. For example, U.S. Pat. No. 6,228,863 to Palermo et al. (with detailed dosage ranges and ratios for opioid analgesics to suitable antagonists; also see U.S. Pat. No. 6,277,384 to Kalko and Colucci) teaches the art of making oral sustained release opioid agonist and antagonist combination product for reducing the abuse potential by patients as well as drug addicts, wherein an analgesically effective amount of an orally active opioid agonist is combined with an opioid antagonist into an oral dosage form which would require a two step extraction process in order to separate the agonist and the amount of antagonist included in the dosage form is sufficient to counteract adverse reactions of acute/chronic use of opioid product if extracted together with the opioid agonist and administered parenterally. However, the opioid agonist and antagonist combinations generally vary significantly in drug solubility and hence need to be processed differently to achieve comparable or similar sustained release characteristics from both components. While doing so, the individual components become distinguishable or else, the full benefits of the controlled release dosage form cannot be obtained.

Recent clinical studies indicate that morphine and most other clinically used opioid analgesics have bimodal inhibitory (analgesic) as well as excitory (hyperanalgesic or anti-analgesic) effects on nociceptive (pain mediating) types of neurons. Opioid analgesia results from activation of inhibitory opioid receptors on neurons in the nociceptive pathways of the peripheral and central nervous systems. The in vitro and in vivo studies contained in U.S. Pat. Nos. 5,472,943; 5,512,578; 5,580,876; 5,585,348; and 5,767,125, all to Crain and Shen (“the Crain patents”) provided the first evidence that simultaneous activation of inhibitory opioid receptors on neurons not only decreases the analgesic potency of morphine and other bimodally acting opioid agonists but underlies development of tolerance, physical dependency, hyperexcitability and other undesirable side-effects that are exacerbated following abnormal supersensitization of excitory opioid receptors on chronic opioid-treated neurons. Selective blockade of excitory, but not inhibitory, opioid receptor functions increases the analgesic potency of morphine, thereby permitting clinical use of reduced dosages of morphine on a sustained basis, while attenuating undesirable side-effects of opioid tolerance/dependence. The Crain patents identified a group of opioid alkaloids and peptides that have remarkably potent blocking actions on excitory, but not inhibitory, opioid receptor functions when administered at appropriately low concentrations to sensory neurons in vitro. At high concentrations, etorphine, dihydroetorphine, and biphalin are potent opioid analgesics. By contrast, at low, subanalgesic concentrations, these opioids have been shown to act as selective antagonists of excitory opioid receptors functions. Furthermore, the clinically used opioid antagonists, naloxone, naltrexone, and nalmefene were shown to have similar heretofore unrecognized properties. At high concentrations, naloxone and naltrexone block both inhibitory (analgesic) and excitory effects of morphine. In contrast, at >1,000-fold lower concentrations, naloxone and naltrexone can selectively block the excitory effects of morphine on sensory neurons and unmask potent inhibitory effects of morphine and other bimodally acting opioid agonists. Furthermore, these studies predicted that appropriately low doses of naloxone or naltrexone which may not only enhance the analgesic potency of morphine and other bimodally acting opioid agonists but may also markedly attenuate their tolerance/dependence liability. In other words, the opioid antagonist enhances the analgesic effect of the agonist, but is aversive in physically dependent human subjects or drug addicts taking about 2-3 times the therapeutically effective dose of the opioid. Subsequent in vivo studies demonstrated that cotreatment with morphine plus ultra-low dose naltrexone, each delivered to the patient in a controlled release manner, does in fact enhance the antinociceptive potency of morphine and attenuate development of withdrawal symptoms in chronic, as well as acute, physical dependence (refer to FIG. 3 in Technology Introduction by S. Crain and K. F. Shen).

The results of in vitro and in vivo animal studies have been confirmed and extended in healthy normal volunteers using a Thermal Sensory Analyzer to apply an electronically controlled series of stimuli. This clinical trial demonstrated that cotreatment with a lower dose of codeine plus a low dose of naltrexone did in fact result in a two-fold increase in the analgesic effect at two hours after drug administration compared to placebo.

As these scientifically proven data show, the plasma concentration of an opioid antagonist must be maintained at a minimal level so as to continue to selectively block the excitory effects of an opioid agonist on sensory neurons over the entire dosing interval, thereby enhancing the analgesic potency of morphine or other bimodally acting opioid agonists. In other words, an oral controlled release capsule dosage form comprising individually processed particles of both opioid agonist and antagonist, releases the agonist and the antagonist at substantially proportionate rates so as to be therapeutically effective over the dosing interval. The opioid agonist and the antagonist may be present in the oral multi-particulate controlled release capsule dosage form as granules, pellets, beads or spheroids coated with dissolution rate controlling polymer blends.

However, the above-cited patents do not teach the art of producing such controlled-release formulations for maintaining the analgesically effective blood levels of agonist during an extended period of dosing, while at the same time maintaining the pharmacologically effective blood levels of the antagonist for reducing the side effects associated with the opioid treatment. The art of making such controlled release compositions to release the opioid agonist and the antagonist at substantially proportionate rates over time more preferably over a dosing period, is disclosed in PCT Application No. WO 01/58447 A1 to B. Oshlack and W. Curtis, thus providing the selective enhancement of analgesic potency of the opioid agonist while attenuating development of physical dependence, tolerance and other undesirable side-effects (e.g., anti-analgesia, hyperalgesia, hyperexcitability) caused by the chronic administration of the opioid agonist. Controlled release solid dosage forms are described that release an opioid agonist and an opioid antagonist over an extended period of time and preferably, the release rates of the two component drugs are approximately proportionate over time, more preferably over the dosing period. To accomplish these objectives, the controlled release formulations of opioid agonist and antagonist are individually processed, drug loading and polymer coating are optimized to provide similar release profiles, and finished dosage forms are produced by combining the two formulations in dose proportionate manner. The dosage form may optionally include, in addition to an opioid agonist and antagonist, one or more drugs that may or may not act synergistically, such as a combination of two agonists differing in elimination half-life, solubility, potency, and hepatic clearance, combination with non-opioid drugs such as aspirin, acetaminophen, NSAIDS (e.g., ibuprofen), COX II inhibitors. The individual agonist and antagonist bead populations in the controlled release morphine/naltrexone dosage forms (capsules) comprise separate bead populations prepared by layering the respective drug on to 30-35 mesh sugar spheres and coating with a controlled release coating. The membrane coated agonist and antagonist bead formulations disclosed in the patent application, can be easily distinguished, and the agonist bead population can be easily separated and thus, the proposed dosage form has high abuse potential.

### SUMMARY OF THE INVENTION

The present invention relates to a method for providing a physical means for camouflaging controlled release (CR) multi-particulate pharmaceutical dosage forms (e.g., beads, pellets, spheroids, or granules presented as tablets or capsules) of opioid agonist and antagonist combinations intended for the treatment of intense pain in patients such that antagonist and agonist bead populations are visually indistinguishable. In particular, the invention is applicable to microencapsulated or coated bead/particle processes whereby combination drug products are comprised of individual bead populations. Combination products based on bead technology are typically combined by separately processing bead batches of an opioid agonist and an opioid antagonist such that these separately processed bead batches not only release the actives at approximately proportionate rates over the entire dosing regimen but also are visually indistinguishable in terms size, shape, appearance and/or color. The invention is particularly useful for formulating combinations of opioid agonist and antagonists for use in pain management therapy whilst avoiding the potential for drug abuse of the agonist component. In conventional combination pharmaceutical dosage forms utilizing bead technology, no attention is paid to the size, shape, appearance and color of individual bead populations, and hence, these bead populations are visually distinguishable by virtue of their shape, size, appearance and/or color. By a proper selection of the size or diameter of neutral cores used for drug layering or the size/diameter of drug containing cores obtained by extrusion—spheronization or wet or dry granulation followed by compression, as well as the thickness of the selected functional polymer coating systems, the controlled release (CR) multi-particles formulated to contain individual components of opioid agonist and antagonist combinations, which are indistinguishable in terms of size and/or shape can be accomplished. One of ordinary skill in the art can readily determine the proper combination of core particle size and coating thickness to achieve bead populations of similar appearance and size. For example, high drug loads can be applied to 20 to 25 mesh spheres and low drug loads can be applied to 16 to 20 mesh spheres to obtain two bead populations of similar final sizes. Furthermore, by proper selection of the dye and its concentration, the final appearance and color of CR multi-particles formulated from different opioid agonist and antagonist combinations can likewise be matched.

In accordance with one embodiment of the present invention, an oral pharmaceutical multi-particulate dosage form comprising at least two populations of beads, a first population of opioid agonist beads and a second population of opioid antagonist beads is provided. The opioid agonist beads provide an analgesically effective amount of opioid agonist and the opioid antagonist beads provide an amount of opioid antagonist effective to attenuate side effects associated with chronic dosing of the opioid agonist. The first population of opioid agonist beads and the second population of opioid antagonist beads are visually indistinguishable, thereby reducing the potential for drug abuse of the opioid agonist by separation of the two bead populations. In accordance with particular aspects of the invention, the opioid agonist beads and opioid antagonist beads are each provided with sustained release membrane coatings capable of releasing the opioid agonist and opioid antagonist at proportionate rates over a dosing interval. The dosing interval may be at least about 8 hours and preferably from about 12-24 hours. The dosage form in accordance with particular embodiments of the present invention may further comprise a non-opioid analgesic, preferably in the form of immediate release (IR) or sustained release (SR) beads.

The present invention is also directed to a method of preparing a pharmaceutical multi-particulate dosage form comprising an opioid agonist population of beads and an opioid antagonist population of beads. In certain embodiments, the method comprises the steps of preparing an opioid agonist core particle, applying a sustained release coating on the opioid agonist core particle, preparing an opioid antagonist core particle, applying a sustained release coating on the opioid antagonist core particle and filling capsules with the opioid agonist and opioid antagonist beads such that the agonist beads will provide an analgesically effective amount of opioid agonist and the opioid antagonist beads provide an amount of opioid antagonist effective to attenuate side effects associated with chronic dosing of the opioid agonist. The agonist beads and antagonist beads are prepared in such a way so as to be visually indistinguishable, thereby reducing the potential for drug abuse of the opioid agonist by separation of the agonist from the opioid antagonist. In accordance with particular embodiments of the present invention, the method may further comprise the step of preparing sustained release non-opioid analgesic beads for inclusion in the multi-particulate dosage form.

### DETAILED DESCRIPTION OF THE INVENTION

All documents cited are, in relevant part, incorporated herein by reference; the citation of any document is not to be construed as an admission that it is prior art with respect to the present invention.

The term “particles” is used generally to refer to individual, discrete particles, irrespective of their size, shape or morphology. Accordingly, the term “particles” includes without limitation such terms as pellets, beads, granules, spheroids, minitabs (minitablets typically 1 to 2 mm in diameter) and these terms are used interchangeably throughout the present application. The term “multi-particulate” as used herein means a plurality of discrete, or aggregated, particles, pellets, beads, granules, spheroids, minitabs or mixture thereof irrespective of their size, shape or morphology.

As used herein, the term “visually indistinguishable” refers to pellets, beads or granules in a multi-particulate dosage form which are derived from more than one population of pellets, beads or granules but for all practical purposes appear to be derived from a single population. The beads, pellets or granules from one population are similar enough in appearance to those from the other population so as to be indistinguishable based on visual examination of the bead populations. The two (or more) populations do not need to be identical with respect to all visual characteristics, but simply need to be similar enough in appearance to make separation of the populations impractical.

As used herein, the term “controlled-release” indicates that the dosage form provides a longer period of pharmacological response after the administration of the agonist and the antagonist than is ordinarily provided after administration of a rapid release dose form.

By “sustained release”, it is meant for purposes of the present application that the release of the therapeutically active agent occurs such that blood levels are maintained within a desired therapeutic range over an extended period of time, e.g., at least about 8 and preferably from about 12 to about 24 hours.

As used herein, the terms “opioid agonist” and “opioid antagonist” include the base, pharmaceutically acceptable salts thereof, stereoisomers thereof, ethers and esters thereof and mixtures thereof.

The active core of the dosage form of the present invention may comprise an inert particle or an acidic or alkaline buffer crystal, which is coated with an opioid agonist- or antagonist-containing film-forming formulation and preferably a water-soluble film forming composition to form a water-soluble/dispersible particle. Alternatively, the active core may be prepared by granulating and milling and/or by extrusion and spheronization of a polymer composition containing opioid agonist or antagonist. Generally, the functional polymeric coating on the active core will be from 1 to 20% based on the weight of the coated particle. Those skilled in the art will be able to select an appropriate amount of opioid agonist or antagonist for coating onto or incorporating into the core to achieve the desired dosage. In one embodiment, the inactive core may be a sugar sphere, a buffer crystal or an encapsulated buffer crystal, such as calcium carbonate, sodium bicarbonate, fumaric acid, tartaric acid, etc. Buffer crystals are useful to alter the microenvironment.

In accordance with one embodiment of the present invention, the water soluble/dispersible drug-containing particle is coated with a sustained release polymer membrane. Examples of useful polymers include water insoluble polymers, combinations of water soluble and water insoluble polymers, or combinations of water insoluble and enteric polymers. The ratio of water insoluble polymer to water insoluble polymer or enteric polymer may vary from 9:1 to 1:1. The membrane thickness varies from about 1% to about 20% and preferably from about 2% to about 10% based on the weight of the coated beads. The polymeric coatings typically contain plasticizers and may be applied from aqueous and/or solvent based systems. Any of the pharmaceutically acceptable food colors can be used in the coating formulation.

The unit dosage form according to certain embodiments of the present invention may comprise an immediate release bead population which provides an immediate release component of an opioid agonist to act as a bolus dose.

The invention also provides a method of making a sustained release dosage form comprising the steps of:

1. individually preparing an active-containing core population (either opioid agonist or antagonist multi-particles) by coating an inert particle such as a non-pareil seed, an acidic buffer crystal or an alkaline buffer crystal, with an opioid agonist or antagonist and a polymeric binder or by granulation and milling or by extrusion/spheronization to form immediate release (IR) beads;

2. individually coating the IR beads population with a plasticized solution or suspension of a sustained release polymer system to form sustained release (SR) coated drug particles;

3. filling into hard gelatin capsules opioid agonist- and antagonist-containing beads in proportion to their doses in the finished dosage forms.

In accordance with the present invention, the desired drug release profiles for the opioid agonist and opioid antagonist are obtained by separately optimizing polymer combinations or coating levels depending on the pH-solubility profiles and pharmacokinetics parameters of the opioid agonist and opioid antagonist. An aqueous or a pharmaceutically acceptable solvent medium may be used for preparing drug containing core particles. The type of film forming binder that is used to bind the agonist or antagonist to the inert sugar sphere is not critical but usually water-soluble, alcohol-soluble or acetone/water soluble binders are used. Binders such as polyvinylpyrrolidone (PVP), polyethylene oxide, hydroxypropyl methylcellulose (HPMC), hydroxypropylcellulose (HPC), polysaccharides, such as dextran, and corn starch may be used at concentrations of from about 0.5 to 10 weight %. The active (agonist or antagonist) may be present in the coating formulation in solution form or may be suspended at a solids content up to about 35 weight % depending on the viscosity of the coating formulation.

Opioid analgesics which are useful in the present invention include all opioid agonists or mixed agonist-antagonists, partial agonists, including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalophine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, mixtures of any of the foregoing, salts of any of the foregoing, and the like.

In certain preferred embodiments, the opioid agonist or analgesic is selected from the group consisting of hydrocodone, morphine, hydromorphone, oxycodone, codeine, levorphanol, meperidine, methadone, salts thereof, and mixtures thereof.

The opioid antagonists particularly useful in the present invention include naloxone, naltrexone, diprenorphine, etorphine, dihydroetorphine, pharmaceutically acceptable salts thereof and mixtures thereof. Other opioid antagonists include nalmefene, cyclazacine, levallorphan, pharmaceutically acceptable salts thereof and mixtures thereof. In certain embodiments, the opioid antagonist is naloxone or naltrexone.

Dissolution rate controlling polymers suitable for incorporating in the formulation for producing granules by high shear or fluid bed granulation or by dry granulation include high molecular weight hydroxypropyl methylcellulose, hydroxypropyl cellulose, ethyl cellulose, sodium carboxymethyl cellulose, alginic acid, polymethylmethacrylate copolymers and polyvinyl acetate/crotonic acid copolymer or combinations thereof.

Acidic buffers, which help maintain an acidic microenvironment within drug containing particles, include fumaric acid, tartaric acid, maleic acid, succinic acid and mixtures thereof. An acidic microenvironment helps dissolve basic drugs with poor solubility at the intestinal pHs and become available for absorption. Examples of alkaline buffers include sodium bicarbonate, calcium carbonate, and sodium dihydrogen phosphate.

An opioid agonist or antagonist, a binder such as PVP, a buffer, a dissolution rate controlling polymer (if used), and optionally other pharmaceutically acceptable excipients are blended together in a high shear granulator such as Fielder or a fluid bed granulator and granulated to form agglomerates by adding/spraying a granulating fluid such as water or alcohol and dried. The wet mass can be extruded and spheronized to produce spherical particles (beads) using an extruder/marumerizer. In these embodiments, the drug load could be as high as 90% by weight based on the total weight of the extruded/spheronized core. The blend can also be used to produce dry granules by slugging in a tablet press or a chilsonator, without the addition of any granulating fluid.

The active containing cores (beads, pellets or granular particles) thus obtained may be coated with one or more layers of polymers to obtain desired release profiles with or without a lag time. The polymer membrane is applied to each of the active containing cores, substantially surrounding each of the core particles. The membrane, which largely controls the rate of release following imbibition of water or body fluids into the core, comprises a water insoluble polymer, such as ethylcellulose, cellulose acetate, polymethylmethacrylate copolymers commercially known as Eudragit RL and RS polymers at a thickness of from 1 to 20% and preferably from 2 to 10% based on the weight of the coated particle. The release rate controlling membrane provided on the drug containing core, may comprise a mixture of a water insoluble polymer and a water soluble polymer or an enteric polymer, at a ratio of 9:1 to 1:1.

Representative examples of water soluble polymers useful in the invention include, but are not limited to, methylcellulose, hydroxypropylcellulose, hydroxypropylmethyl-cellulose and polyvinylpyrrolidone.

Representative examples of enteric polymers useful in the invention include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methamethacrylate copolymers and shellac. These polymers may be used as a dry powder or an aqueous dispersion. Some commercially available materials that may be used are methacrylic acid copolymers sold under the trademark Eudragit (L100, S100, L30D) manufactured by Rhom Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K. K.

The coating polymers used in forming the membranes are usually plasticized. Representative examples of plasticizers that may be used to plasticize the membranes include triacetin, tributyl citrate, triethyl citrate, acetyl tri-n-butyl citrate diethyl phthalate, castor oil, dibutyl sebacate, acetylated monoglycerides and the like or mixtures thereof. The plasticizer may comprise about 3 to 30 wt. % and more typically about 10 to 25 wt. % based on the polymer. The type of plasticizer and its content depends on the polymer or polymers, nature of the coating system (e.g., aqueous or solvent based, solution or dispersion based and the total solids).

In general, it is desirable to prime the surface of the particle before applying the rate controlling release membrane coatings or to separate the different membrane layers by applying a thin hydroxypropyl methylcellulose (HPMC) (Opadry® Clear, Opadryo® White Opaque or colored Opadry®) film. While HPMC is typically used, other primers such as hydroxypropylcellulose (HPC) can also be used.

The membrane coatings can be applied to the core using any of the coating techniques commonly used in the pharmaceutical industry, but fluid bed coating is particularly preferred. The present invention is applied to multi-dose forms, i.e., drug products in the form of multi-particulate dosage forms (pellets, beads, granules or mini-tablets) or in other forms suitable for oral administration.

The composition of the coating formulation and/or coating levels on agonist and antagonist bead populations are optimized so as to maintain an analgesically effective amount of the opioid agonist in the blood throughout the dosing period and to maintain the concentration of the opioid antagonist in the blood throughout the dosing period sufficient for decreasing the side effects associated with the opioid agonist, such as drug dependence, but not sufficient to negate the analgesic efficacy of the agonist. Preferably, the release rates of the opioid agonist and antagonist are maintained to be approximately proportionate over the dosing period. Under such a scenario, the opioid antagonist binds to and inactivates excitatory receptors on neurons in the nociceptive pathways, thereby enhancing the analgesic effects of the opioid agonist. The finished dosage form (capsules) may include controlled release beads of two opioid agonists having different pharmacokinetic properties, such as half-life, solubility, potency, and a combination of any of the forgoing and an opioid antagonist. The finished dosage form (capsules) may also include controlled release beads of an opioid agonist, an opioid antagonist, and a non-opioid analgesic. The non-opioid analgesic may be present in the form of IR or SR beads. Examples of such non-opioid analgesics include, for example, aspirin, acetaminophen, non-steroidal anti-inflammatory drugs (NSAIDS), N-methyl-D-Aspartate (NMDA) receptor antagonists, cycooxygenase-II inhibitors (COX-II inhibitors); and/or glycine receptor antagonists. Specific examples include acetaminophen, celecoxib, dextromethorphan, and ibuprofen. Combinations of non-opioid analgesics may also be used. Additional non-opioid analgesics are described in U.S. Pat. No. 6,228,863.

The drug release profiles from combination product capsules comprising sustained release bead populations can be determined using a USP Apparatus 1 (baskets at 100 rpm) or Apparatus 2 (paddles at 50 rpm) in 900 mL purified water, pH 1.2 or pH 6.8 or 7.5 or using a two-stage dissolution medium (first 2 hours in 700 mL 0.1N HCl at 37° C. followed by dissolution at pH=6.8 obtained by the addition of 200 mL of pH modifier). Drug release with time is determined by HPLC on samples pulled at selected intervals. For purposes of the present invention, drug release profiles provide an indication of drug delivery in vivo.

The present invention can be described in greater detail by reference to the following, non-limiting, examples.

### EXAMPLE 1

Morphine Sulfate SR Beads: Morphine sulfate (4 kg) is slowly added to an aqueous solution of polyvinylpyrrolidone (200 g Povidone K-30) and mixed well. 25-30 mesh sugar spheres (4.4 kg) are coated with the drug solution in a fluid bed granulator. The drug containing pellets are dried, and a seal coat of Opadry Clear (200 g) is first applied. The polymer coating is applied to the active particles (9.0 kg) by spraying a solution of ethylcellulose (640 g) and diethyl phthalate (160 g) in 98/2 acetone/water. An outer coating of Opadry White Opaque (200 g) is applied on the functionally coated beads. The beads are cured in an oven at 60° C. for 4 hours.

Naltrexone HCl SR Beads: Naltrexone hydrochloride (60 g) is slowly added to an aqueous solution of mannitol (500 g) and hydroxypropylcellulose (Klucel LF 200 g) and mixed well. 18-22 mesh sugar spheres (5.0 kg) are coated with the drug solution in a fluid bed granulator. The drug containing pellets are dried, and a seal coat of Opadry Clear (140 g) is first applied. The polymer coating is applied to the active particles (5.9 kg) by spraying a solution of ethylcellulose (450 g) and hydroxypropylcellulose (Klucel LF 150 g) in 85/15 acetone/water. An outer coating of Opadry White Opaque (200 g) is applied on the functionally coated beads. The beads are cured in an oven at 60° C. for 4 hours.

Controlled Release Morphine Sulfate/Naltrexone Hydrochloride Capsules, 100 mg/600 μg, are produced by filling 250 mg morphine sulfate SR beads and 69 mg naltrexone hydrochloride SR beads into hard gelatin capsules using a capsule filling equipment. The SR beads of morphine sulfate and naltrexone HCl exhibit similar (proportionate) extended release profiles over a 12 hour period. The SR beads in the capsule product are visually indistinguishable.

### EXAMPLE 2

Oxycodone HCl SR Beads: Oxycodone hydrochloride (2.5 kg) is slowly added to an aqueous solution of mannitol (950 g) and polyvinylpyrrolidone (400 g Povidone K-30) and mixed well. 20-30 mesh sugar spheres (3.5 kg) are coated with the drug solution in a fluid bed granulator. The drug containing pellets are dried, and a seal coat of Opadry Clear (150 g) is first applied. The polymer coating is applied to the active particles (7.5 kg) by spraying a solution of ethylcellulose (600 g) and hydroxypropylcellulose (150 g) in 85/15 acetone/water. An outer coating of Opadry White Opaque (150 g) is applied on the functionally coated beads. The beads are cured at 60° C. for 10-30 minutes while moderately fluidizing the beads in the fluid bed equipment.

Naltrexone HCl SR Beads: Naltrexone hydrochloride (60 g) is slowly added to an aqueous solution of mannitol (500 g) and hydroxypropylcellulose (Klucel LF 200 g) and mixed well. 20-25 mesh sugar spheres (5.0 kg) are coated with the drug solution in a fluid bed granulator. The drug containing pellets are dried, and a seal coat of Opadry Clear (140 g) is first applied. The polymer coating is applied to the active particles (5.9 kg) by spraying a solution of ethylcellulose (450 g) and hydroxypropylcellulose (Klucel LF 150 g) in 85/15 acetone/water. An outer coating of Opadry White Opaque (200 g) is applied on the functionally coated beads. The beads are cured at 60° C. for 10-30 minutes while moderately fluidizing the beads in the fluid bed equipment.

Controlled Release Oxycodone HCl/Naltrexone Hydrochloride Capsules, 50 mg/1 mg, are produced by filling 115 mg naltrexone hydrochloride SR beads and 168 mg oxycodone HCl SR beads into hard gelatin capsules using a capsule filling equipment. The SR beads of oxycodone HCl and naltrexone HCl exhibit similar (proportionate) extended release profiles over a 12 hour period. The SR beads in the capsule product are visually indistinguishable.

### EXAMPLE 3

Oxycodone HCl SR Beads: Oxycodone hydrochloride (2.5 kg) is slowly added to an aqueous solution of mannitol (950 g) and polyvinylpyrrolidone (400 g Povidone K-30) and mixed well. 20-30 mesh sugar spheres (3.5 kg) are coated with the drug solution in a fluid bed granulator. The drug containing pellets are dried, and a seal coat of Opadry Clear (150 g) is first applied. The polymer coating is applied to the active particles (7.5 kg) by spraying a solution of ethylcellulose (600 g) and hydroxypropylcellulose (150 g) in 85/15 acetone/water. An outer coating of Opadry Clear (150 g) containing 200 mg of FD&C Blue No.1 Aluminum Lake was applied on the functionally coated beads. The beads are cured in an oven at 60° C. for 4 hours.

Naltrexone HCl SR Beads: Naltrexone hydrochloride (60 g) is slowly added to an aqueous solution of mannitol (500 g) and hydroxypropylcellulose (Klucel LF 200 g) and mixed well. 20-25 mesh sugar spheres (5.0 kg) are coated with the drug solution in a fluid bed granulator. The drug containing pellets are dried, and a seal coat of Opadry Clear (140 g) is first applied. The polymer coating is applied to the active particles (5.9 kg) by spraying a solution of ethylcellulose (450 g) and hydroxypropylcellulose (Klucel LF 150 g) in 85/15 acetone/water. An outer coating of Opadry Clear (200 g) containing 100 mg of FD&C Blue No.1 Aluminum Lake is applied on the functionally coated beads. The beads are cured in an oven at 60° C. for 4 hours.

Controlled Release Oxycodone HCl/Naltrexone Hydrochloride Capsules, 25 mg/300 μg, are produced by filling 34.5 mg naltrexone hydrochloride SR beads and 84 mg oxycodone HCl SR beads into hard gelatin capsules using a capsule filling equipment. The SR beads of oxycodone HCl and naltrexone HCl exhibit similar (proportionate) extended release profiles over a 12 hour period. The SR beads in the capsule product are visually indistinguishable.

Many modifications can be made to the illustrated examples by those skilled in the art without exceeding the scope or departing from the spirit of the claimed invention.

What is claimed is:

1. An oral pharmaceutical multi-particulate dosage form comprising at least two populations of particles, a first population of opioid agonist particles and a second population of opioid antagonist particles wherein said opioid agonist particles provide an analgesically effective amount of opioid agonist and said opioid antagonist particles provide an amount of opioid antagonist effective to attenuate side effects associated with chronic dosing of said opioid agonist, said first population of opioid agonist particles and said second population of opioid antagonist particles being visually indistinguishable, thereby reducing the potential for drug abuse of said opioid agonist by separation of said opioid agonist from said opioid antagonist.

2. A dosage form as defined in claim 1 wherein said opioid agonist particles comprise a core comprising an opioid agonist and a sustained release membrane coating substantially surrounding said core;
said opioid antagonist particles comprise a core comprising an opioid antagonist and a sustained release membrane coating substantially surrounding said core;
said sustained release membrane providing for sustained drug release;
wherein the dosage form provides for release of the opioid agonist and opioid antagonist at proportionate rates over a dosing interval.

3. A dosage form as defined in claim 2, wherein said sustained release membrane coating comprises a water insoluble polymer, a combination of a water insoluble polymer and a water soluble polymer at a ratio of about 9:1 to about 1:1 or a combination of a water insoluble polymer and an enteric polymer wherein said membrane has a thickness of from about 1% to about 20% based on weight of the coated beads.

4. A dosage form as defined in claim 3 wherein said water insoluble polymer is selected from the group consisting of ethylcellulose, cellulose acetate, polymethylmethacrylate copolymers and mixtures thereof.

5. A dosage form as defined in claim 3 wherein said water soluble polymer is selected from the group consisting of methylcellulose, hydroxypropylcellulose, hydroxypropylmethylcellulose, and polyvinyl pyrrolidone.

6. A dosage form as defined in claim 3 wherein the enteric polymer is selected from the group consisting of cellulose acetate phthalate, hydroxypropyl methylcellulose phthalate, polyvinyl acetate phthalate and mixtures thereof.

7. A dosage form as defined in claim 1 wherein said opioid agonist is selected from the group consisting of hydrocodone, morphine, hydromorphone, oxycodone, codeine, levorphanol, meperidine, methadone, salts thereof, and mixtures thereof.

8. A dosage form as defined in claim 1 wherein said opioid antagonist is selected from the group consisting of naloxone, naltrexone, etorphine, pharmaceutically acceptable salts thereof and mixtures thereof.

9. A dosage form as defined in claim 1 further comprising a non-opioid analgesic.

10. A dosage form as defined in claim 9 further comprising a third population of particles comprising said non-opioid analgesic.

11. A dosage form as defined in claim 10 wherein said non-opioid analgesic is selected from the group consisting of aspirin, acetaminophen, non-steroidal anti-inflammatory drugs (SAIDS), dextromethorphan, COX-II inhibitors and combinations thereof.

12. A dosage form as defined in claim 1 wherein said opioid agonist comprises morphine sulfate and said opioid antagonist comprises naltrexone hydrochloride.

13. A dosage form as defined in claim 1 wherein said opioid agonist comprises oxycodone hydrochloride and said opioid antagonist comprises naltrexone hydrochloride.

14. A dosage form as defined in claim 2 wherein said dosing interval is at least 12 hours.

15. A dosage form as defined in claim 2 wherein said agonist core comprises a non-pareil seed coated with said opioid agonist in a polymeric binder and said antagonist core comprises a non-pareil seed coated with said opioid antagonist in a polymeric binder.

16. A method of preparing a pharmaceutical multi-particulate dosage form comprising a first population of particles comprising an opioid agonist and a second population of particles comprising an opioid antagonist, wherein said method comprises the steps of:
(a) preparing opioid agonist cores by layering an aqueous solution comprising said opioid agonist and a binder on non-pareil seeds;
(b) applying a sustained release coating on said opioid agonist cores, said sustained release coating comprising a water insoluble polymer or a combination of a water insoluble polymer and a water soluble polymer to produce a first population of particles comprising said opioid agonist;
(c) preparing opioid antagonist cores by layering an aqueous solution comprising said opioid antagonist and a binder on non-pareil seeds;
(d) applying a sustained release coating on said opioid antagonist cores, said sustained release coating comprising a water insoluble polymer or a combination of a water insoluble polymer and a water soluble polymer to produce a second population of particles comprising said opioid antagonist; and
(e) filling capsules with said first population of particles and said second population of particles wherein said opioid agonist particles provide an analgesically effective amount of opioid agonist and said opioid antagonist particles provide an amount of opioid antagonist effective to attenuate side effects associated with chronic dosing of said opioid agonists, said first population of particles and said second population of particles being visually indistinguishable, thereby reducing the potential for drug abuse of said opioid agonist by separation of said opioid agonist from said opioid antagonist.

17. The method of claim 16 wherein said sustained release coating comprises ethylcellulose.

18. The method of claim 16 wherein said water soluble polymer is selected from the group consisting of methylcellulose, hydroxypropylcellulose, hydroxypropylmethylcellulose, and polyvinylpyrrolidone.

19. The method of claim 16 wherein said opioid agonist is selected from the group consisting of hydrocodone, morphine, hydromorphone, oxycodone, codeine, levorphanol, meperidine, methadone, salts thereof, and mixtures thereof.

20. The method of claim 16 wherein said opioid antagonist is selected from the group consisting of naloxone, naltrexone, etorphine, pharmaceutically acceptable salts thereof and mixtures thereof.

21. The method of claim 16 wherein said pharmaceutical multi-particulate dosage form further comprises a third population of particles comprising a non-opioid analgesic, said method further comprising the steps of:
(f) preparing non-opioid analgesic cores by layering an aqueous solution comprising said non-opioid analgesic and a binder on non-pareil seeds;
(g) applying a sustained release coating on said non-opioid analgesic cores, said sustained released coating comprising a water insoluble polymer or a combination of a water insoluble polymer and a water soluble polymer; and
(h) incorporating said non-opioid analgesic particles into said pharmaceutical multi-particulate dosage form.

22. The method of claim 21 wherein said non-opioid analgesic is selected from the group consisting of aspirin, acetaminophen, non-steroidal anti-inflammatory drugs (NSAIDS), dextromethorphan, COX-II inhibitors and combinations thereof.

23. The method of claim 16 wherein said opioid agonist comprises morphine sulfate and said opioid antagonist comprises naltrexone hydrochloride.

24. The method of claim 16 wherein said opioid agonist comprises oxycodone hydrochloride and said opioid antagonist comprises naltrexone hydrochloride.
